SEQUENCE LISTING (7 sequences)

SEQ ID NO: 1 - DNA, 2282 bp, Homo sapiens
aattcacacatcccaataattatctttcaacatgatcactctaggaagctctctactaattctgacaatatgttcacaataccagcttgagatgttgcctcagataagtatctttggtttcaaatgcgttaatatgatctgggtcgataatccttgttttagaaacttgtatccctttttggttttcctatattatctaagtcatcaaacttattgacatatatttactcataatgtttagttattatatcatattttctgtaggatctatagtaacattctcttaatcatttctgacattgtgttttcctctgtattgtcttgatgaattttgctagagatgtatcaattttcttcatctttttataaaactaggttttggcttttgttgactttatcatttactcttaactttattaatttatattcttgacattattttttatcttctgtagttgactcttaaacaatgtgagtttgaacatacgggtccacttacgtacaaatattcttctgcctctgtcatctcgagacagaaggactaaccattccctttcctcctcctcctcagcctactcaacataaagatgatgaggatgaagatgtttatgatgatcacttctacttaataaagaataactatattttttattctgtgtgattgtcttaactttctctatcttactttattgtaagaatagagtatatatatatatatagcataaaaatatggattaacagactatttatgttattggtaatacatactttgtcataactttaactgagtaggtgaaagatctgtacactggaaattacaaaaaattgatgaaagaaattaaacaaggcacaaataaattgaaagatgtcttgtgtttccagatttaaatagttaatattgttaaatgtccatagggattcctttttatcctcttcacttctcatcgcttgctgtgctggcctatgcctctgtctgtgtgtctcatcaaaagccatatgttaatctcaacagatgcagaaaaagcttttgagaaattcaaaaattgcttcacgataaaaactctcaactcgttaggcacaaaggaacacgcctcaaaaggaacacgcctcaaaataatatgagctatctatgacaaactcacagccaacatcatactgaatgggcaaaagctggaagtgttccccttaagaattagaataagacaaggatgcccactctcaccactcctgttcaactggaagtcctaggcagagcaatcaggaaagagaaagaaataaaaggcatccatataggaaaagaagaagttaaatagtctctcttcactgatgttataattttatacctagggaaccctaaagattctgccaaaataatcctagagctgataaaacaattttactaaagtttcaggttacaaaatcatgtacaaaaatcactagcatttctgaacactgataatgttcaaactgggaaccaaagcaaaacgcaattccatagccacacaaaaactaaaatacctaggaataaatcaaatcaaggaggtgaaagatctctccaaggagagctgcaaaacactattgaaaaaagtcagagatgatacaaacaaatggaaaaaaattccatgttcatggattggaagaattagtgttgtaaaatggccaaactacccaaagcagccagcacgattcaactctattcctatcaaattaccaacatcacttttcacagaattaggggaaactattctaaaattcatatggaaccaaaaaagagcctgaatagccaaggcaatcctaagcataaagctggaggcgtcacattacccaacttcaaactatactacaaggcaaaacagtatagtactggtatgaaggcagacacatacatgaatggaacatgatagagaacccagaaataaaaccacacacctacagtcatctgatcttcaacaaagttgacaaaaacataagcaatacagtaagtactctatagtcaataaatggcactgcaatagctggctatccatatgcagaagaatgaaactggactcttgcttaaaaccatatacaaaaattagctcaagatggattaaagacttaaatgtaagatctaaaactataaaaatcctaggagaaaacataagaaatatgcttctatacatttacattggtcttggcaaaaaaattataactaagtcctcaaaagcaactgcaacaaaaatgaaaactgacaaaagatgtctaattaaactaaagagcttctgcacagcaaaacaaactat

SEQ ID NO: 2 - DNA, 50 bp, Homo sapiens
aatatgatctgggtcgataatccttgttttagaaacttgtatcccttttt

SEQ ID NO: 3 - DNA, 50 bp, Homo sapiens
tccacttacgtacaaatattcttctgcctctgtcatctcgagacagaagg

SEQ ID NO: 4 - DNA, 50 bp, Homo sapiens
cctatgcctctgtctgtgtgtctcatcaaaagccatatgttaatctcaac

SEQ ID NO: 5 - DNA, 230 bp, Homo sapiens
cctcaaaaggaacacgcctcaaaataatatgagctatctatgacaaactcacagccaacatcatactgaatgggcaaaagctggaagtgttccccttaagaattagaataagacaaggatgcccactctcaccactcctgttcaactggaagtcctaggcagagcaatcaggaaagagaaagaaataaaaggcatccatataggaaaagaagaagttaaatagtctctct

SEQ ID NO: 6 - DNA, 30 bp, Homo sapiens
taaaatacctaggaataaatcaaatcaagg

SEQ ID NO: 7 - DNA, 460 bp, Homo sapiens
actctattcctatcaaattaccaacatcacttttcacagaattaggggaaactattctaaaattcatatggaaccaaaaaagagcctgaatagccaaggcaatcctaagcataaagctggaggcgtcacattacccaacttcaaactatactacaaggcaaaacagtatagtactggtatgaaggcagacacatacatgaatggaacatgatagagaacccagaaataaaaccacacacctacagtcatctgatcttcaacaaagttgacaaaaacataagcaatacagtaagtactctatagtcaataaatggcactgcaatagctggctatccatatgcagaagaatgaaactggactcttgcttaaaaccatatacaaaaattagctcaagatggattaaagacttaaatgtaagatctaaaactataaaaatcctaggagaaaacataagaaatat

(19) United States
(12) Patent Application Publication
(10) Pub. No.: US 2016/0199279 A1
(43) Pub. Date: Jul. 14, 2016

(54) METHODS OF MAKING PERSONAL CARE COMPOSITIONS COMPRISING ANIMAL DNA

(71) Applicant: Jacobus Reyneke, Upper Saddle River, NJ (US)

(72) Inventor: Jacobus Reyneke, Upper Saddle River, NJ (US)

(21) Appl. No.: 14/595,524

(22) Filed: Jan. 13, 2015

(51) Int. Cl. A61K 8/60

(52) U.S. Cl. CPC A61K 8/606; A61Q 1/06

(57) ABSTRACT

Disclosed herein are methods of making personal care compositions comprising animal DNA. The DNA may be extracted from animal tissues or cultured animal cells, or synthesized chemically or enzymatically. The personal care compositions may include shampoos, lotions, cosmetics, and the like. Also disclosed herein are personal care compositions comprising animal DNA prepared according to the methods disclosed herein.

### REFERENCE TO A SEQUENCE LISTING

This application contains references to nucleic acid sequences which have been submitted concurrently herewith as the sequence listing text file “61409779_1.txt”, file size 51 KiloBytes (KB), created on 6 Jan. 2015. The aforementioned sequence listing is hereby incorporated by reference in its entirety pursuant to 37 C.F.R. §1.52(e)(5).

### FIELD

This invention relates generally to methods for making personal care compositions comprising animal DNA, as well as to the resulting compositions made according to such methods.

### BACKGROUND

This section provides background information related to the present disclosure which is not necessarily prior art.

US 2007/0218549 to Mansbridge reports personal care compositions comprising proteins and small molecule effluents of cultured cells, including cultured human cells. These compositions include shampoos and cosmetics. Some of the cultured cells contain recombinant DNA, including recombinant human DNA.

Both SEDAL and YVES ROCHER have marketed personal care compositions containing plant DNA. SEDAL has produced a shampoo that contains unspecified amounts of an unspecified plant DNA. YVES ROCHER has produced creams and lotions that contain an unspecified amount of wheat germ DNA.

### SUMMARY

This section provides a general summary of the disclosure, and is not a comprehensive disclosure of its full scope or all of its features.

Disclosed herein are methods of making a composition for topical administration and personal care compositions comprising animal DNA prepared by the methods. In certain embodiments, the animal DNA is mammalian DNA, such as canine, feline, or human DNA.

In an embodiment, there is provided a method of making a composition for topical administration, the method comprising: mixing water, animal-derived deoxyribonucleic acid (animal DNA), and at least one compound of Formula (I)

CH₃—(CH₂)ₙ—X (I)

or a salt thereof, to form a substantially homogenous mixture,

wherein n is an integer from 7 to 21, inclusive, and

wherein X has a molecular weight less than or equal to 500 daltons and is a moiety selected from the group consisting of alcohol, amide, amine, carbonyl, carboxyl, ester, ether, hydroxyl, phosphonyl, and sulfonyl, and

wherein the composition comprises at least 5 wt % compound of Formula (I) and at least 0.1 wt % animal DNA based on total mass of the composition.

In another embodiment, there is provided a composition for topical administration produced by the method described above.

Also disclosed herein are personal care compositions comprising animal DNA. In certain embodiments, the animal DNA is mammalian DNA, such as canine, feline, or human DNA. In certain embodiments the animal DNA comprises sequences derived from NCBI Genome ID Nos. 51, 78, or 85. In certain embodiments the animal DNA comprises sequences derived from SEQ ID NO:1.

In certain embodiments, the animal DNA is produced by culturing animal cells in vitro and extracting DNA from them. In other embodiments, the animal DNA is synthesized chemically or enzymatically, such as by polymerase chain reaction (PCR).

In certain embodiments, the compositions disclosed herein are lotions, creams, gels (including hydrogels), powders, serums, salves, foundations, facial masks, lip care products (such as lipsticks or lip balms), sunscreens, hair care products (such as shampoos, conditioners, including deep conditioners, hair care treatments, hot oil treatments, and the like), skin cleansers, exfoliants, compact formulations, eye-liners, or mascaras. In certain embodiments, the compositions further include detergents.

### DETAILED DESCRIPTION

### Glossary

All scientific and technical terms take their ordinary meaning unless otherwise specified. Each technical and scientific term used herein has the same meaning each time it is used. It must also be noted that as used herein and in the appended claims, the singular forms “a,” “an,” and “the” include plural referents unless the context clearly dictates otherwise. The use of “or” in a listing of two or more items indicates that any combination of the items is contemplated, for example, “A or B” indicates that A alone, B alone, or both A and B are intended.

As used herein, the term “animal DNA” refers to deoxyribonucleic acid extracted from an animal cell or synthesized to conform to a sequence originally extracted from an animal cell. With regard to enzymatically synthesized DNA, one of skill in the art will appreciate that even high-fidelity polymerases do not always copy nucleotide sequences with 100% fidelity, and that therefore the term “animal DNA” should be understood also to include sequences that show small and insignificant variation from sequences isolated from primary animal cells.

In a similar manner to “animal DNA,” the term “human DNA” refers to DNA derived from a human organism, the term “feline DNA” refers to DNA derived from a feline organism, the term “canine DNA” refers to DNA derived from a canine organism, etc. and so forth. It should be understood that “human organism” as used herein refers not only to recognizably human individuals, but also to organs, tissues, and cells derived from humans at any stage of development from zygote, to embryo, to fetus, to mature adult. The term “human organism” also should be understood to include human clones and chimeras, such as a hybridoma cell line created by the fusion of a human cell with a non-human (e.g., murine or hamster) cell. The creation of hybridomas is well understood in the art. See, e.g., U.S. Pat. No. 5,552,286 to Chang et al.

### In Vitro Cell Culture

In certain embodiments, personal care compositions comprising animal DNA can be manufactured according to the following method: (1) obtain animal cells from an organism of interest; (2) culture these cells in vitro; (3) extract DNA from a portion of the cultured cells; (4) mix the extracted DNA with water and a fatty acid or fatty acid derivative until a substantially homogenous personal care composition is formed. The fatty acid or fatty acid derivative can be a compound according to Formula (I):

CH₃—(CH₂)ₙ—X (I)

or a salt thereof, wherein n is an integer from 7 to 21, inclusive, and wherein X has a molecular weight less than or equal to 500 daltons and is a moiety selected from the group consisting of alcohol, amide, amine, carbonyl, carboxyl, ester, ether, hydroxyl, phosphonyl, and sulfonyl.

Techniques for isolating and culturing animal cells are well known in the art. See, e.g., Jeffrey W. Pollard & John M. Walker, BASIC CELL CULTURE PROTOCOLS (1997). Media and other supplies for tissue culture are also known in the art and available for purchase. Methods of extracting DNA are well known in the art. See, e.g., Joseph Sambrook, E. F. Fritsch, & Tom Maniatis, MOLECULAR CLONING: A LABORATORY MANUAL (1989). Supplies, reagents, and equipment for DNA extraction are also known in the art and available for purchase.

In certain embodiments, the cultured cell can be an immortalized cell. The immortalization of cell lines is well understood in the art, and can be achieved by any of a number of well-known techniques. See, e.g., Harris et al. (1995) J. Bone. Miner. Res. 10:178-86. In certain embodiments, the cultured cell can be a hybridoma.

### Chemical or Enzymatic Synthesis of DNA

In certain embodiments, personal care compositions comprising animal DNA can be manufactured according to the following method: (1) synthesize DNA to correspond in sequence with DNA that can be isolated from an animal; (2) mix the extracted DNA with water and a fatty acid or fatty acid derivative until a substantially homogenous personal care composition is formed. The fatty acid or fatty acid derivative can be a compound according to Formula (I):

CH₃—(CH₂)ₙ—X (I)

or a salt thereof, wherein n is an integer from 7 to 21, inclusive, and wherein X has a molecular weight less than or equal to 500 daltons and is a moiety selected from the group consisting of alcohol, amide, amine, carbonyl, carboxyl, ester, ether, hydroxyl, phosphonyl, and sulfonyl.

DNA synthesis can be performed using chemical and or enzymatic methods. Both chemical and enzymatic synthesis methods are well known in the art. See, e.g. Caruthers (1991) Acc. Chem. Res. 24(9):278-84 and Erlich (1989) J. Clin. Immunol. 9(6):437-47. Most enzymatic DNA synthesis methods require a template starting material from which copies can be made. The template for use in these methods can be obtained by the techniques described above for extraction of DNA from animal cells. Additionally or alternatively, material copied by enzymatic synthesis or chemically synthesized de novo can serve as the template for enzymatic synthesis.

### Fatty Acids and Fatty Acid Derivatives

There are many common and commercially available fatty acids and fatty acid derivatives according to Formula (I) that may be used in methods and compositions described herein. In certain embodiments, the compound according to Formula (I) for use in methods and compositions described herein may have n values equaling 7, 9, 11, 13, 15, 17, 19, or 21. In certain embodiments, compositions described herein may include a mix of compounds according to Formula (I), wherein n=11, 13, 15, or 17. By way of non-limiting example, the compound according to Formula (I) may be selected from the group consisting of disodium stearoyl, ethylhexyl palmitate, glyceryl stearate, glycol distearate, hexyl laurate, lauramidopropyl betaine, laureth sulfate, lauryl sulfate, 2-octyldodecyl myristate, propylene glycol laurate, propylene glycol stearate, propylheptyl caprylate, sorbitan laurate, and stearyl alcohol.

The compound according to Formula (I) can supply at least about 1 wt % of the total weight of the personal care composition, for example at least about 2 wt %, at least about 3 wt %, at least about 4 wt %, at least about 5 wt %, at least about 10 wt %, at least about 15 wt %, at least about 20 wt %, at least about 25 wt %, at least about 30 wt %, at least about 35 wt %, or at least about 40 wt % of the total weight of the personal care composition. Ranges expressly disclosed include any combination of the above-enumerated weight percentages, e.g. about 5-30 wt %, about 3-20 wt %, about 1-15 wt %, etc.

### DNA

DNA for use in the methods and composition described herein should comprise a sequence or set of sequences that derive ultimately from an organism of the biological kingdom Animalia, for example an organism of the biological phylum Chordata and the biological class Mammalia. As noted above, this DNA can be extracted from animal cells, or synthesized by means of any of a variety of chemical or enzymatic methods.

In certain embodiments the animal DNA is derived from a mammalian organism such as a cat, a dog, or a human. For example, DNA for use in methods and compositions as described herein can comprise at least about 100 bp, at least about 200 bp, at least about 500 bp, at least about 1 kb, at least about 5 kb, at least about 10 kb, at least about 50 kb, at least about 100 kb, or at least about 500 kb of contiguous nucleotides from NCBI Genome ID Nos. 51, 78, or 85. In certain embodiments the DNA may derive from a pet cat or pet dog. In certain embodiments, the DNA can comprise at least 10, at least 15, at least 20, at least 50, at least 100, or at least 500 contiguous nucleotides from SEQ ID NO:1 (human specific DNA sequence). Non-limiting examples of DNA sequences that may be used in the methods and compositions disclosed herein include any one or more of SEQ ID NOs:2-7. In certain embodiments, the DNA is derived from a human individual whose name or likeness is familiar or recognizable to at least five hundred thousand, at least one million, at least five million, at least ten million, at least one hundred million, or at least five hundred million other contemporaneously living humans. In certain embodiments the DNA is sonicated or otherwise fractured by some mechanical process.

Compositions as described herein can include at least about 0.01 wt % animal DNA, for example at least about 0.02 wt %, at least about 0.03 wt %, at least about 0.04 wt %, at least about 0.05 wt %, at least about 0.06 wt %, at least about 0.07 wt %, at least about 0.08 wt %, at least about 0.09 wt %, at least about 0.1 wt %, at least about 0.2 wt %, at least about 0.3 wt %, at least about 0.4 wt %, at least about 0.5 wt %, at least about 0.6 wt %, at least about 0.7 wt %, at least about 0.8 wt %, at least about 0.9 wt %, at least about 1 wt %, at least about 2 wt %, at least about 3 wt %, at least about 4 wt %, at least about 5 wt %, at least about 6 wt %, at least about 7 wt %, at least about 8 wt %, at least about 9 wt %, at least about 10 wt %, at least about 15 wt %, or at least about 20 wt % relative to the total weight of the composition. Ranges expressly disclosed include any combination of the above-enumerated weight percentages, e.g., about 0.02-10 wt %, about 0.01-0.5 wt %, about 0.01-0.05 wt %, about 1-20 wt %, etc.

### Compositions

The personal care compositions disclosed herein may be suitable for topical administration, for example administration to skin or hair. In certain embodiments, the compositions disclosed herein are lotions, creams, gels (including hydrogels), powders, serums, salves, foundations, facial masks, lip care products (such as lipsticks or lip balms), sunscreens, hair care products (such as shampoos, conditioners, including deep conditioners, hair care treatments, hot oil treatments, and the like), skin cleansers, exfoliants, compact formulations, eye-liners, or mascaras. Each exemplary composition above can be formulated with conventional materials known to a person having ordinary skill, and a skilled artisan understands that various combinations of the conventional materials are possible. In certain embodiments, the inventive compositions include detergents.

The compositions disclosed herein can be made according to the methods described above. In certain embodiments, the composition can be a shampoo or body wash. In this case, the composition should comprise at least one detergent. Suitable detergents include but are not limited to lauramidopropyl betaine, laureth sulfate, and lauryl sulfate. Shampoos or body washes as disclosed herein may optionally include a co-surfactant, a salt (e.g., sodium chloride), a preservative, a dye, or a fragrance. Other ingredients may be optionally included, particularly ingredients known in the art to affect qualities such as foaming, ease of rinse, skin/eye irritation, viscosity, biodegradability, acidity/alkalinity, or repair of minor damage to hair. In addition, tiny flakes of pearlescent materials, e.g. glycol distearate, may be included to create a shiny appearance, or silicone to “condition” hair. Shampoos or body washes as disclosed herein may include one or more of the following ingredients: ammonium chloride; ammonium lauryl sulfate; a botanical extract, citric acid; di-PPG-2 myreth-10 adipate; glycol; methylisothiazolinone; polysorbate 20 (PEG 20); PEG 80; PEG 150 distearate; polyquaternium-10; quaternium-15; sodium laureth sulfate; sodium lauroamphoacetate; or sodium lauryl sulfate.

Shampoos are typically formulated at pH 5.5 because slightly acidity causes the scales on a hair follicle to lie flat. This creates a smooth and look shiny appearance for hair.

In certain embodiments, the composition can be a lotion. Lotions as disclosed herein may be oil-in-water emulsions or water-in-oil emulsions. The lotion may optionally contain an emulsifier, e.g., cetearyl alcohol. Optionally, a lotion as disclosed herein may also contain a wide variety of other ingredients such as an antimicrobial, a dye, a fragrance, glycerol, petroleum jelly, a preservative, a protein, and a stabilizing agent.

In certain embodiments, the composition can be a cosmetic, such as a foundation, lipstick, or mascara. These compositions typically contain one or more dyes or pigments. Suitable fatty acid derivatives for use in a cosmetic include but are not limited to caprylic triglyceride, ethylhexyl palmitate, glyceryl stearate, hexyl laurate, octyldodecyl myristate, propylheptyl caprylate, propylene glycol stearate, and sorbitan laurate. Cosmetics as disclosed herein may also optionally contain a wax or oil to affect the texture or shine of the cosmetic.

### Further Embodiments

Additionally or alternately, the present invention can include one or more of the following embodiments.

### Embodiment 1

A method of making a composition for topical administration, the method comprising: mixing water, animal-derived deoxyribonucleic acid (animal DNA), and at least one compound of Formula (I):

CH₃—(CH₂)ₙ—X (I)

or a salt thereof, to form a substantially homogenous mixture,

wherein n is an integer from 7 to 21, inclusive, and

wherein X has a molecular weight less than or equal to 500 daltons and is a moiety selected from the group consisting of alcohol, amide, amine, carbonyl, carboxyl, ester, ether, hydroxyl, phosphonyl, and sulfonyl, and

wherein the composition comprises at least 5 wt % compound of Formula (I) and at least 0.1 wt % animal DNA based on total mass of the composition.

### Embodiment 2

The method of Embodiment 1, wherein the compound of Formula (I) is selected from the group consisting of disodium stearoyl, ethylhexyl palmitate, glyceryl stearate, glycol distearate, hexyl laurate, lauramidopropyl betaine, laureth sulfate, lauryl sulfate, 2-octyldodecyl myristate, propylene glycol laurate, propylene glycol stearate, propylheptyl caprylate, sorbitan laurate, and stearyl alcohol.

### Embodiment 3

The method of any one of the previous embodiments, further comprising extracting DNA from an animal cell.

### Embodiment 4

The method of any one of the previous embodiments, wherein the animal cell is an immortalized cell.

### Embodiment 5

The method of any one of the previous embodiments, wherein the animal is selected from the group consisting of cat, dog, and human.

### Embodiment 6

The method of any one of the previous embodiments, wherein the animal is a human.

### Embodiment 7

The method of any one of the previous embodiments, wherein the composition further comprises a fragrance or dye.

### Embodiment 8

The method of any one of the previous embodiments, wherein the composition is a cosmetic selected from the group consisting of eye-liner, foundation, lipstick, lotion, and mascara.

### Embodiment 9

The method of any one of the previous embodiments, wherein the compound of Formula (I) is a detergent, and wherein the composition is a shampoo or body wash that comprises between about 5 wt % and about 30 wt % of the compound of Formula (I) based on total mass of the composition.

### Embodiment 10

The method of Embodiment 1 or Embodiment 2, further comprising amplifying the DNA by polymerase chain reaction.

### Embodiment 11

The method of any one of the previous embodiments, wherein the DNA comprises at least 100 contiguous base pairs from any one of NCBI Genome ID Nos. 51, 78, and 85.

### Embodiment 12

The method of Embodiment 11, wherein the at least 100 contiguous base pairs are selected from SEQ ID NO:1.

### Embodiment 13

The method of Embodiment 10, wherein the composition further comprises a fragrance or dye.

### Embodiment 14

The method of any one of the previous embodiments, wherein the composition is a cosmetic selected from the group consisting of eye-liner, foundation, lipstick, lotion, and mascara.

### Embodiment 15

The method of any one of the previous embodiments, wherein the compound of Formula (I) is a detergent, and wherein the composition is a shampoo or body wash that comprises between about 5 wt % and about 30 wt % of the compound of Formula (I) based on total mass of the composition.

### Embodiment 16

The method of any one of the previous embodiments, wherein the composition comprises at least about 2 wt % animal DNA based on total mass of the composition.

### Embodiment 17

A composition for topical administration produced by any method of Embodiments 1-16.

### Embodiment 18

The composition of Embodiment 17, wherein the compound of Formula (I) is selected from the group consisting of disodium stearoyl, ethylhexyl palmitate, glyceryl stearate, glycol distearate, hexyl laurate, lauramidopropyl betaine, laureth sulfate, lauryl sulfate, 2-octyldodecyl myristate, propylene glycol laurate, propylene glycol stearate, propylheptyl caprylate, sorbitan laurate, and stearyl alcohol.

### Embodiment 19

The composition of Embodiment 17 or Embodiment 18, wherein the composition is a cosmetic selected from the group consisting of eye-liner, foundation, lipstick, lotion, and mascara.

### Embodiment 20

The composition of Embodiment 17 or Embodiment 18, wherein the compound of Formula (I) is a detergent, and wherein the composition is a shampoo or body wash that comprises between about 5 wt % and about 30 wt % of the compound of Formula (I) based on total mass of the composition.

### EXAMPLES

The following examples are merely illustrative, and do not limit this disclosure in any way.

### Example 1

In Example 1 blood is drawn from a famous musician and hybridomas are created from B lymphocytes in this blood sample. The hybridomas are cultured and at periodic intervals DNA is extracted from hybridoma cells.

A shampoo is manufactured by mixing water, sodium lauryl sulfate, sodium laureth sulfate, sodium chloride, glycol distearate, laureth-4, sodium citrate, sodium xylenesulfonate, dimethicone, rosemary extract, citric acid, sodium benzoate, tetrasodium EDTA, polyquartenium-6, methylchloroisothiazolinone, methylisothiazolinone and 0.1 wt % human DNA extracted from the hybridomas in a high speed mixer. The ingredients are mixed overnight at 4 krpm at 15° C. until the composition has achieved a homogenous consistency. The resulting shampoo mixture is bottled and stored for use.

### Example 2

In Example 2 a body wash, eye-liner, foundation, lipstick, lotion, or mascara comprising DNA from a famous musician is produced in a similar manner by using the DNA extraction method described in Example 1.

### Example 3

In Example 3 blood is drawn from a pet dog and DNA is extracted from the blood sample. Portions of this DNA are amplified by PCR using primers specific for canine sequences.

A lotion is manufactured by mixing glycerin, petrolatum, dicapryl ether, dimethicone glyceryl stearate, sweet almond extract, PEG 30 stearate, tocopheryl acetate, C₁₀₋₃₀ alkyl acrylate cross-polymers, dimethiconol, methylparaben, propylparaben, glyceryl acrylate, and propylene glycol overnight at 4 krpm at 30° C. At the same time, cetyl alcohol, benzyl alcohol, disodium EDTA, sodium hydroxide, and the PCR-produced canine DNA are dissolved in enough water to equal twice the mass of the combined lipids mix. The DNA is added in a quantity sufficient to equal 0.1 wt % of the aqueous and lipid mixtures combined. The lipid mixture is blended into the aqueous mix over the course of twelve hours in a mixer at 4 krpm at 20° C. The resulting lotion is bottled and stored for use.

### Example 4

In Example 4 blood is drawn from a pet cat and DNA is extracted from the blood sample. Portions of this DNA are amplified by PCR using primers specific for feline sequences.

A foundation is manufactured by mixing ethylhexyl palmitate, talc, kaolin, neopentyl glycol diethylhexanoate, silica, octodecyl myristate, propylhexyl caprylate, microcrystalline wax, ozokerite, hydrogenated castor oil, lecithin, sorbitan sesquioleate, phenoxyethanol, propylene glycol stearate, ethylhexylglycerin, sorbic acid, tocopheryl acetate, polysorbate 20, propylene glycol laurate, sorbitan laurate, tocopherol, 0.1 wt % PCR-produced feline DNA, and bismuth oxychloride. The ingredients are blended over the course of twelve hours in a mixer at 4 krpm at 20° C. The resulting cosmetic is packaged and stored for use.

The above examples are strictly exemplary, and should not be construed to limit the scope or understanding of the present invention. It should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the described invention. All such modifications are intended to be within the scope of the claims appended hereto. The publications discussed herein are provided solely for their disclosure prior to the filing date of the present application. Nothing herein is to be construed as an admission that the described invention is not entitled to antedate such publication by virtue of prior invention. Further, the dates of publication provided may be different from the actual publication dates which may need to be confirmed independently.

What is claimed is:

1. A method of making a composition for topical administration, the method comprising:
mixing water, animal-derived deoxyribonucleic acid (animal DNA), and at least one compound of Formula (I)
CH₃—(CH₂)ₙ—X (I)
or a salt thereof, to form a substantially homogenous mixture,
wherein n is an integer from 7 to 21, inclusive, and
wherein X has a molecular weight less than or equal to 500 daltons and is a moiety selected from the group consisting of alcohol, amide, amine, carbonyl, carboxyl, ester, ether, hydroxyl, phosphonyl, and sulfonyl, and
wherein the composition comprises at least 5 wt % compound of Formula (I) and at least 0.1 wt % animal DNA based on total mass of the composition.

2. The method of claim 1, wherein the compound of Formula (I) is selected from the group consisting of disodium stearoyl, ethylhexyl palmitate, glyceryl stearate, glycol distearate, hexyl laurate, lauramidopropyl betaine, laureth sulfate, lauryl sulfate, 2-octyldodecyl myristate, propylene glycol laurate, propylene glycol stearate, propylheptyl caprylate, sorbitan laurate, and stearyl alcohol.

3. The method of claim 2, further comprising extracting DNA from an animal cell.

4. The method of claim 3, wherein the animal cell is an immortalized cell.

5. The method of claim 4, wherein the animal is selected from the group consisting of cat, dog, and human.

6. The method of claim 5, wherein the animal is a human.

7. The method of claim 6, wherein the composition further comprises a fragrance or dye.

8. The method according to claim 7, wherein the composition is a cosmetic selected from the group consisting of eye-liner, foundation, lipstick, lotion, and mascara.

9. The method of claim 7, wherein the compound of Formula (I) is a detergent, and
wherein the composition is a shampoo or body wash that comprises between about 5 wt % and about 30 wt % of the compound of Formula (I) based on total mass of the composition.

10. The method of claim 2, further comprising amplifying the DNA by polymerase chain reaction.

11. The method of claim 10, wherein the DNA comprises at least 100 contiguous base pairs from any one of NCBI Genome ID Nos. 51, 78, and 85.

12. The method of claim 11, wherein the at least 100 contiguous are selected from SEQ ID NO:1.

13. The method of claim 12, wherein the composition further comprises a fragrance or dye.

14. The method of claim 13, wherein the composition is a cosmetic selected from the group consisting of eye-liner, foundation, lipstick, lotion, and mascara.

15. The method of claim 13, wherein the compound of Formula (I) is a detergent, and
wherein the composition is a shampoo or body wash that comprises between about 5 wt % and about 30 wt % of the compound of Formula (I) based on total mass of the composition.

16. The method of claim 2, wherein the composition comprises at least about 2 wt % animal DNA based on total mass of the composition.

17. A composition for topical administration produced by the method of claim 1.

18. The composition of claim 17, wherein the compound of Formula (I) is selected from the group consisting of disodium stearoyl, ethylhexyl palmitate, glyceryl stearate, glycol distearate, hexyl laurate, lauramidopropyl betaine, laureth sulfate, lauryl sulfate, 2-octyldodecyl myristate, propylene glycol laurate, propylene glycol stearate, propylheptyl caprylate, sorbitan laurate, and stearyl alcohol.

19. The composition of claim 18, wherein the composition is a cosmetic selected from the group consisting of eye-liner, foundation, lipstick, lotion, and mascara.

20. The composition of claim 18, wherein the compound of Formula (I) is a detergent, and
wherein the composition is a shampoo or body wash that comprises between about 5 wt % and about 30 wt % of the compound of Formula (I) based on total mass of the composition.
